(19) United States
(12) Patent Application Publication
(10) Pub. No.: US 2003/0105404 A1
(43) Pub. Date: Jun. 5, 2003

(54) Multi-parameter acquisition of ECG and other physiologic data exclusively employing conventional ECG lead conductors

(76) Inventors: Peter M. Galen, McMinnville, OR (US); Martin Baumer, Carlton, OR (US)

Correspondence Address: KOLISCH HARTWELL, P.C., 520 S.W. YAMHILL STREET, SUITE 200, PORTLAND, OR, 97204, US

(21) Appl. No.: 10/175,352

(22) Filed: Jun. 18, 2002

Related U.S. Application Data
(60) Provisional application No. 60/299,265, filed on Jun. 18, 2001.
(60) Provisional application No. 60/299,161, filed on Jun. 18, 2001.
(60) Provisional application No. 60/299,264, filed on Jun. 18, 2001.
(60) Provisional application No. 60/299,580, filed on Jun. 19, 2001.
(60) Provisional application No. 60/299,577, filed on Jun. 19, 2001.
(60) Provisional application No. 60/299,550, filed on Jun. 19, 2001.
(60) Provisional application No. 60/299,551, filed on Jun. 19, 2001.
(60) Provisional application No. 60/299,522, filed on Jun. 20, 2001.

(51) Int. Cl. A61B 5/04

(52) U.S. Cl. 600/509

(57) ABSTRACT

Method and apparatus employing transducers for collection multi-parameter ECG-related data and for supplying all categories of that data exclusively, and in certain different ways, over only conventional ECG lead conductors.

### CROSS-REFERENCE TO RELATED APPLICATIONS

This application claims the priority under 35 U.S.C. §119 and applicable foreign and international law of the following U.S. Provisional Patent Applications: Serial No. 60/299,265 filed Jun. 18, 2001, No. 60/299,161 filed Jun. 18, 2001, No. 60/299,264 filed Jun. 18, 2001, Serial No. 60/299,580 filed Jun. 19, 2001, Serial No. 60/299,577 filed Jun. 19, 2001, Serial No. 60/299,550 filed Jun. 19, 2001, Serial No. 60/299,551 filed Jun. 19, 2001, and Serial No. 60/299,522 filed Jun. 19, 2001, all of which are hereby incorporated by reference in their entireties for all purposes.

### BACKGROUND AND SUMMARY OF THE INVENTION

This invention relates to structure and methodology for gathering and communicating multiple-parameter data from a subject for the purpose of evaluating and observing heart condition and functionality. The different categories of data always include ECG data, and at least one additional category of data, such as phono/sound data, and spatial-orientation data. The invention is designed whereby it can readily handle more than one category of other-than-ECG data if so desired.

Very specifically, the invention focuses attention on the gathering of such data by a suitably constructed sensor device which includes appropriate data transducers, and the subsequent communication this data to the outside world, such as to a cardiograph device, and/or to an ECG-data interpretation structure, utilizing only conventional ECG lead conductors Among the special features that are offered by the invention are the following:

(1) the gathering of multiple-parameter, ECG-related, physiologic data, and the transmitting of that data outwardly to external structure utilizing solely ECG lead-conductors to perform such transmitting;

(2) the gathering and transmitting of data, generally in the categories mentioned above, wherein data relating to plural parameters is transmitted to the outside world utilizing only a single, conventional ECG lead conductor which is appropriately electrically referenced to a subject's anatomy; and

(3) the transmission of gathered data in such a fashion that each category of gathered and transmitted ECG-related data is transmitted to the outside world over a conventional ECG lead conductor, wherein the electrical signal on that conductor is referenced specifically to another such lead conductor.

In each of these feature categories, the necessity for providing and using additional wiring, that is, wiring outside of ECG lead conductors, is completely avoided.

These and other features and advantages that are offered by the present invention will become more fully apparent as the description which now follows is read in conjunction with the accompanying drawings.

### BRIEF DESCRIPTION OF THE DRAWINGS

FIGS. 1, 2 and 3 are simplified block/schematic diagrams which illustrate, respectively, one each of the three above-listed features and advantages offered by the invention. Very specifically, FIG. 1 generally illustrates the concept of utilizing only ECG lead conductors for the outward communication of multiple-parameter ECG-related physiologic data.

FIG. 2 illustrates, generally, the gathering and collection of ECG and other-parameter data, and then the transmitting of this data over a single ECG lead conductor, appropriately referenced.

FIG. 3 pictures schematically the feature of the invention which, in the setting of utilizing only ECG lead conductors for outward transmission of data, each category of data is transmitted outwardly over an ECG lead conductor with respect to which electrical signal-voltage referencing is made in relation to another, conventional ECG lead conductor.

FIG. 4 is a block/schematic diagram illustrating an ECG-related data gathering system which employs the present invention. In this system, there is pictured a specific structural organization that bears the responsibility for gathering data, with this organization including a two-part device. This two-part device includes what is referred to herein as an adaptor, and this adaptor is directly connected to conventional ECG lead conductors. It also includes a disconnectable sensor which includes the desired data-gathering transducers. Such a two-part construction may, if desired, be replaced by but a single, integrated device.

FIG. 5 is a circuit diagram of one form of two-part device such as that generally referred to above in the description of FIG. 4.

FIG. 6 is another circuit diagram illustrating specifically an adaptor/sensor organization which functions, with respect to gathered data, to transmit all of that data outwardly over but a single, conventional ECG lead conductor (appropriately referenced).

### DETAILED DESCRIPTION, AND BEST MODE FOR CARRYING OUT, THE INVENTION

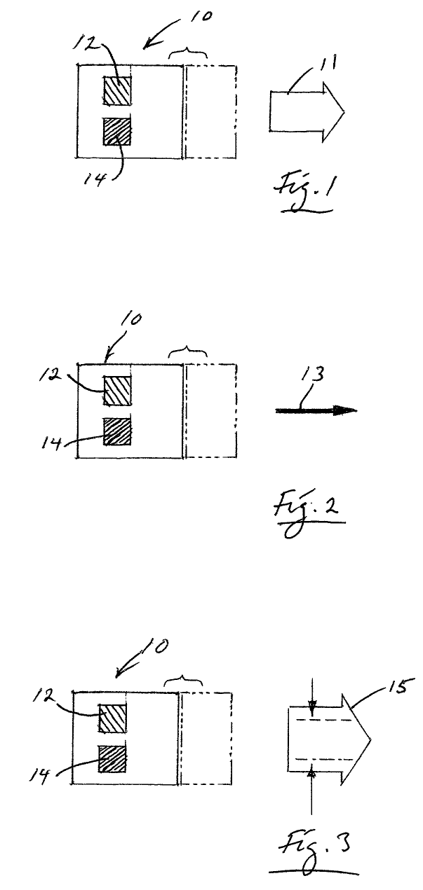

Turning attention now to the drawings, and referring first of all to FIG. 1, here at 10, there is illustrated a single-part (integrated) data collector (shown in solid lines, or selectively a two-part data collector (add the component pictured in dash-triple-dot lines), which is provided with plural data-gathering transducers, such as the two shown at 12, 14. These two transducers are pictured with different shadings to highlight the fact that they are designed to collect different categories of ECG-related data. Transducer 12 functions to collect ECG data, and transducer 14 certain other-parameter data, such as phono/sound data. The large data-flow outwardly pointing arrow 11 in FIG. 1 represents the fact of operation, according to the invention, whereby only standard ECG lead conductors are employed for outward transmission of data.

In FIG. 2, there is also shown at 10, a single-unit sensor (in solid lines) constructed in accordance with the invention, and including the same two data-collection transducers 12, 14 that are pictured in FIG. 1. The slender dark single arrow 13 which points outwardly and to the right in FIG. 2 represents that feature of a modification of the invention which offers the opportunity to transmit outwardly-going data on a single ECG lead conductor (properly electrically referenced).

FIG. 3 shows at 10 yet another single-part sensor (solid lines) constructed in accordance with the invention, including the same two data-gathering transducers 12, 14. Pointing outwardly to the right of sensor 10 in FIG. 3 is a large data arrow 15 within which there appeared two short horizontal lines, one of which is illustrated in dash-dot fashion, and the other in dash-double-dot fashion. These two lines are shown generally parallel to one another, and vertically spaced. Two vertical, opposite-direction arrows point toward one another and touch a different one of these two lines, respectively, to represent the offering made by the present invention of a situation where all ECG and other-parameter gathered data is communicated on a conventional ECG lead conductor which is referenced electrically to another conventional ECG lead conductor.

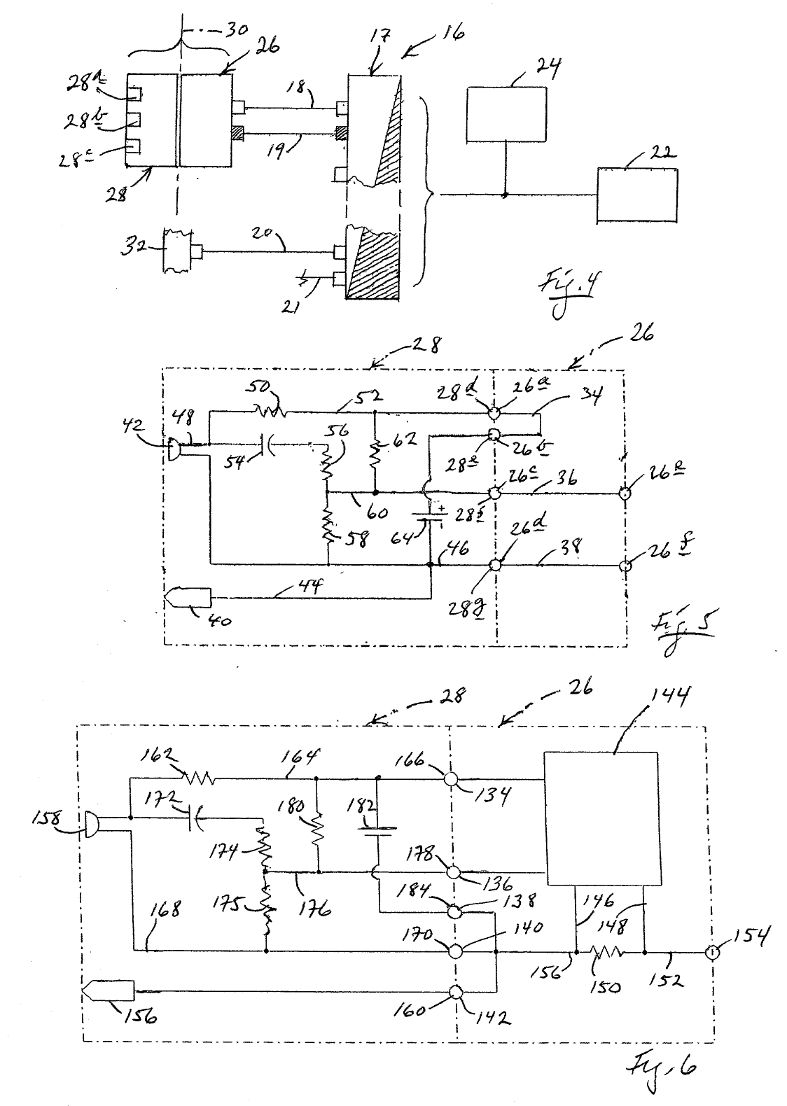

Turning attention now to FIG. 4, here illustrated generally at 16 is a system employing the present invention to gather, in addition to conventional ECG data, one or more other categories of data, such as sound data and spatial-orientation data. Centered in FIG. 4 as a fragmented block 17 is a collection of conventional ECG lead conductors. This block is shaded diagonally to highlight the fact that various different conventional styles of ECG lead conductors can be employed. The shaded area, for example, represents one set of conventional lead conductors, and the unshaded portion of block 17 represents another set. On the left side of this block in FIG. 4 are shown some small rectangles which represent points of connection for outwardly extending conductor leads, four of which are shown at 18, 19, 20 and 21 in FIG. 4.

Illustrated on the right side of FIG. 4, appropriately connected to the output side of block 17, are a conventional cardiograph machine 22, and, just for illustration purposes, a data-interpretation unit 24. The exact constructions of these two structures (22, 24) form no part of the present invention, and thus are neither illustrated nor discussed in any detail herein.

As shown in FIG. 4, conductor leads 18, 19 extend from block 17 to a block 26 which functions as an adaptor herein. Block 26 is also referred to herein as a first part.

Disconnectably connected to adaptor 26 is a sensor which is shown in block form at 28. Adaptor 26 and sensor 28 are coupled through appropriate physical interface conductor structure (not specifically shown in FIG. 1) with connection between these two units effectively residing in a region of joinder which is represented by dash-dot line 30 in FIG. 1.

While discussion regarding FIG. 4 relates to a two-part (26, 28) data-collector herein, the connected assembly of these two blocks functions like the single-block device pictured in FIGS. 1, 2 and 3.

Included within sensor 28 in the illustration now being given are three transducers 28a, 28b, 28c which are employed to gather different kinds of data in accordance with practice of the invention. Transducer 28a is designed to collect sound data, transducer 28b conventional ECG data, and transducer 28c spatial-orientation data. Such data, in accordance with the present invention, and in its entirety, is communicated outwardly from the connected adaptor and sensor utilizing only conventional ECG lead conductors, such conductors 18, 19 mentioned above.

Lead conductor 20 is shown connected to a fragmentary illustration at 32 which is intended to represent the right leg of a subject. This right-leg connection is most frequently employed in the gathering of ECG data to act as a reference point electrically with regard to the collection of ECG data. It should be noted, and it is known, that the so-called right-leg reference could be replaced with another type of connection. The specific site for such a reference connection herein is not part of the present invention.

The other leads, only one of which is shown at 21, that form part of lead conductors 17 extend appropriately for connection to other points on the anatomy of a subject where data is being collected.

One will note that, in FIG. 4, two terminal connectors relevant to a lead-conductor interconnection between the pictured adaptor unit and block 17 have been darkened. This is intended to reflect the fact that this connection can be employed as a singularity, according to one manner of structuring and practicing the invention, to convey all data which is communicated to the outside world over just a single lead conductor. Data signals on this singular conductor are referenced electrically to the lead conductor (20) which extends to the so-called right leg site pictured at 32 in FIG. 4.

Focusing attention now on what is shown in FIG. 5, adaptor 26 contains internal conductor circuitry including conductors 34, 36, 38. Conductor 34 bridges between two adaptor terminals shown at 26a, 26b in FIG. 5. Conductor 36 extends between two adaptor terminals 26c, 26e that are provided in adaptor 26. Conductor 38 extends between and connects two adaptor terminals 26d, 26f also provided in adaptor 26.

Included within sensor 28 are an ECG electrode, or transducer, 40, and an appropriate microphone, or transducer, 42. These two transducers are located on and with respect to the body of sensor 28 in such a manner that they can appropriately “engage” a selected local site on the surface of a subject's body.

Electrode 40 is connected through a conductor 44, and a conductor 46, to a sensor terminal 28g. Terminal 28g connects with terminal 26d (as shown) when the adaptor and sensor are interconnected as pictured in FIG. 5. The two output sides of microphone 42 are connected as shown, with the lower side being directly connected to conductor 46, and the upper side being connected through a conductor 48, a resistor 50, and a conductor 52 to a sensor terminal 28d. Terminal 28d connects directly with previously-mentioned terminal 26a in adaptor 26 when the adaptor and sensor are interconnected as shown in FIG. 5.

Extending between conductor 52 and a conductor 60 is a resistor 62. The right end of conductor 60 in FIG. 5 connects to a sensor terminal 28f, and the left end of conductor 60 connects to the junction that exists between two more resistors shown at 56, 58. Terminal 28f connects directly with previously mentioned terminal 26c in adaptor 26 under circumstances, such as is pictured in FIG. 5, with the sensor and adaptor interconnected. The lower end of resistor 58 connects with previously-mentioned conductor 46, and the upper end of resistor 56 connects through a filter capacitor 54 to the upper side of microphone 42 in FIG. 5.

Completing a description of what is shown in FIG. 5, an electrical power source in the form of a battery 64 is furnished within the confines of the body of sensor 28. The positive terminal of this battery is connected to a sensor terminal 28e provided in the sensor, and the negative side of the battery is connected directly to previously mentioned conductor 46. Terminal 28e is directly connected to previously mentioned terminal 26b in adaptor 26 when the adaptor and sensor are connected as shown in FIG. 5.

With circuitry in place as such is pictured in FIG. 5, one can see that conventional ECG data is furnished through terminal 26f to a conventional ECG lead conductor, such as previously-mentioned conductor 18, and that phonic data is supplied to another conventional ECG lead conductor, such as previously-mentioned conductor 19 through terminal 26e. Thus, all information from plural parameters which is communicated ultimately to the outside world is furnished, in accordance with the invention, completely through otherwise entirely conventional ECG lead conductors.

Focusing attention now on FIG. 6 which shows a modified form of adaptor/sensor, adaptor 26 includes a set of interface terminals 134, 136, 138, 140, 142 which establish effective connections, as will be explained, to circuitry contained in sensor 28. Included in the adaptor is a conventional voltage-controlled oscillator/modulator 144 which, as can be seen, has terminals connecting directly through appropriate conductors to terminals 134, 136. Oscillator/modulator 144 also is connected through a pair of conductors 146, 148 to the opposite sides of an output resistor 150 whose right end is connected through a conductor 152 to yet another terminal 154 in the adaptor. The left end of resistor 150 is connected through a conductor 156 to a fan-out arrangement of conductors which effectively connects conductor 156 to each of terminals 138, 140, 142 in the adaptor. It is important to note at this juncture that on the right side of adaptor 26, as such is pictured in FIG. 6, there is but a single conductor terminal, which terminal is connected to a single ECG lead conductor (not shown). As will be explained and will become apparent, it is over this single lead conductor, functioning as a single output conductor from the assembled adaptor and sensor in FIG. 6, that all parameter data, including ECG data, are communicated in the system ultimately to the outside world.

Continuing the description of what is shown in FIG. 6, disposed within the body of sensor 28 are an electrode, or transducer, 156 which is adapted to collect ECG information, and a microphone, or transducer, 158, which is designed to collect phono data. Electrode 156 is directly connected as shown to a terminal 160 in the sensor which is coupled to previously-mentioned terminal 142 in the adaptor.

The upper side of microphone 158 in FIG. 6 is connected through a resistor 162 and a conductor 164 to a terminal 166 in the sensor. Terminal 166, as can be seen, is coupled to terminal 134 in the adaptor.

The lower side of microphone 158 in FIG. 6 is connected through a conductor 168 to a terminal 170 in the sensor, which terminal is connected, as shown, to previously mentioned terminal 140 in the adaptor. Connecting the upper side of microphone 158 in FIG. 6 to conductor 168 is a filter/gain circuit including a capacitor 172 and a voltage divider including resistors 174, 175. The connection region between resistors 174, 175 is connected to a conductor 176 which extends to and connects with yet another terminal 178 in sensor 28. Terminal 178 is electrically connected, as shown, to terminal 136 in the adaptor. Interconnecting conductors 164, 176 in the sensor is a resistor 180.

Completing a description of what is shown in FIG. 6, at 182 there is provided a battery whose positive terminal is connected to conductor 164, and whose negative terminal is connected directly, as shown, to still another terminal 184 in the sensor. Terminal 184 couples to previously mentioned terminal 138 in adaptor 26.

When adaptor 26 and sensor 28 are connected as shown in FIG. 6, and placed into use, both ECG data and phonic data are collected at a localized anatomical site, and are both delivered to terminal 154 on the right side of the adaptor in FIG. 6. ECG data flows directly through resistor 150 to terminal 154. Phono data is fed initially through terminals 134, 136 to oscillator/modulator 144. The oscillator/modulator feeds a suitably modulated rendition of the phono data across resistor 150, and thence to terminal 154. Thus, it is the case that ECG data and phono data are effectively combined for output through single terminal 154. The combined ECG and phono signals, though combined and delivered to a single ECG conductor, are electrically distinguishable for later separation, as by demodulation.

It will thus be apparent that the present invention offers all of the advantages ascribed to it earlier herein. Specifically, it offers all of the advantages that relate to utilizing, in different ways, only conventional ECG lead conductors to communicate multi-parameter data gathered from a subject.

While the invention has been disclosed in a particular setting, and in particular forms herein, the specific embodiments disclosed, illustrated and described herein are not to be considered in a limiting sense. Numerous variations, some of which have been discussed, are possible. Applicants regard the subject matter of their invention to include all novel and non-obvious combinations and subcombinations of the various elements, features, functions and/or properties disclosed herein. No single feature, function, element or property of the disclosed embodiments is essential. The following claims define certain combinations and subcombinations which are regarded as useful, novel and non-obvious. Other such combinations and subcombinations of features, functions, elements and/or properties may be claimed through amendment of the present claims or through presentation of new claims in this or in a related application. Such amended and/or new claims, whether they are broader, narrower or equal in scope to the originally presented claims, are also regarded as included within the subject matter of applicants' invention.

We claim:

1. Apparatus adapted for operative connection to a conventional ECG lead conductors for collecting, from a subject, plural-parameter physiologic data, including ECG data, said apparatus comprising
subject-proximity, plural-parameter, data-gathering sensor structure operatively attachable to conductors in a group of such ECG lead conductors, and operable to gather associated physiologic data from a subject in the form of parameter-differentiable electrical signals, and
electrical circuitry operatively connected to said sensor structure and operable, with the apparatus operatively connected to such a lead conductors, to cause each such parameter differentiable signal to be delivered to and to flow in a conduction path which includes at least one of the ECG lead conductors.

2. The apparatus of claim 1, wherein said electrical circuitry is constructed whereby it causes both gathered ECG data, at least one category of gathered other-parameter data, to flow in a common and shared conductor in the ECG lead conductors.

3. The apparatus of claim 1, wherein said electrical circuitry is structured whereby each component of sensor-gathered data flows in an ECG lead conductor which is voltage referenced to another ECG lead conductor.

4. A method of acquiring from a subject, and outwardly conveying multi-parameter ECG-related data which includes both ECG data and other-parameter data comprising
gathering such data from one or more anatomical sites in a subject utilizing appropriate transducers, and
outwardly conveying all such data solely on lead conductors in an otherwise conventional group of ECG lead conductors.
